(19) United States
(12) Patent Application Publication
(10) Pub. No.: US 2014/0303559 A1
(43) Pub. Date: Oct. 9, 2014

(54) AUTOMATIC SYRINGE PUMPS FOR DRUG AND FLUID DELIVERY

(71) Applicant: WILLIAM MARSH RICE UNIVERSITY, Houston, TX (US)

(72) Inventors: Jinwoo Peter Jung, Mission, TX (US); Lemuel Ming-Jun Soh, Houston, TX (US); Glenn Fiedler, Austin, TX (US); Kevin J. Jackson, Mobile, AL (US); Lavanya Rao, Buffalo, NY (US); Ken Hackenberg, Houton, TX (US); Zaid Haque, Bethesda, MD (US); April Kuo-Ann Kwan, Round Rock, TX (US); Pablo Andres Henning, Houston, TX (US)

(21) Appl. No.: 14/245,854

(22) Filed: Apr. 4, 2014

Related U.S. Application Data
(60) Provisional application No. 61/809,013, filed on Apr. 5, 2013.

(51) Int. Cl. A61M 5/145

(52) U.S. Cl. CPC A61M 5/1454; A61M 2005/14506
USPC 604/135

(57) ABSTRACT

An automatic syringe pump is disclosed that includes at least four subsystems: mechanical power, electronic timing, mechanical power transfer, and user interface subsystems. In some embodiments, an occlusion detection subsystem is also present. The mechanical power subsystem's main includes a constant force spring capable of storing the energy needed to reliable depressed several different sizes of a syringe. The electronic timing subsystem may include components including a ratchet, pawls, flippers, stepper motor, and microcontroller. These components are used together to regulated the release of the energy stored in the constant force spring. The mechanical power transfer subsystem may include components including a rack and pinion which translates the rotational motion of the drive shaft from the constant force spring into linear motion to depress the syringe. Finally, the user interface subsystem may include components including the microcontroller, Arduino backpack, and syringe tray.

### CROSS-REFERENCE TO RELATED APPLICATION

This application claims priority to and the benefit of Provisional Application No. 61/809,013, entitled “AUTOMATIC SYRINGE PUMPS FOR DRUG AND FLUID DELIVERY”, filed Apr. 5, 2013, which is herein incorporated by reference in its entirety for all purposes.

### BACKGROUND

Syringe pumps have been created in the developed world, but existing syringe pumps are typically not available or feasible for use in low resource settings.

### SUMMARY

The present disclosure relates to an automatic syringe pump that is powered by a constant force spring. In one embodiment, the constant force spring stores energy that causes rotation of a driveshaft. The rotation of the driveshaft is restricted by a ratchet and two pawl system. While the pawl is engaged with the ratchet, there will not be rotation of the driveshaft and as a result, no syringe depression. To allow for syringe depression, the pawls are disengaged one at a time in an alternating fashion, which allows for slight rotations of the driveshaft. The disengagement of the pawls is controlled by a stepper motor, which is powered by a microcontroller. The depression of the syringe occurs as a result of the rotation of the driveshaft. When the driveshaft rotates, a pinion gear located on the driveshaft traverses a rack. As the pinion moves along the rack, the depression cage moves as well and depresses the syringe. In one embodiment, an occlusion sensing mechanism (such as a potentiometer-based occlusion sensing mechanism) is also provided.

### BRIEF DESCRIPTION OF THE DRAWINGS

FIG. 1. depicts a generalized view of an overall design of an automatic syringe pump displaying the power system, timing system, and user interface, in accordance with aspects of the present disclosure;

FIG. 2 depicts a generalized timing system, in accordance with aspects of the present disclosure;

FIG. 3 depicts a generalized user interface, in accordance with aspects of the present disclosure;

FIG. 4 depicts a generalized power system, in accordance with aspects of the present disclosure;

FIG. 5 depicts a further view of an automatic syringe pump, in accordance with aspects of the present disclosure;

FIG. 6 depicts an additional view of an automatic syringe pump, in accordance with aspects of the present disclosure;

FIG. 7 depicts a further view of a mechanical power system, in accordance with aspects of the present disclosure;

FIG. 8 depicts a further view of an electronic timing system, in accordance with aspects of the present disclosure;

FIG. 9, depicts a schematic view of the operation of the pawls and ratchet of the timing system, in accordance with aspects of the present disclosure;

FIG. 10 depicts a view of a mechanical power transfer system, in accordance with aspects of the present disclosure;

FIG. 11 depicts a further view of a user interface, in accordance with aspects of the present disclosure;

FIG. 12 depicts an additional view of a user interface, in accordance with aspects of the present disclosure.

FIG. 13 depicts a potentiometer for use with an automatic syringe pump, in accordance with aspects of the present disclosure;

FIG. 14 is an exploded view of the layers of one example of a potentiometer for use in accordance with aspects of the present disclosure;

FIG. 15 depicts an example implementation of a potentiometer in use with an automatic syringe pump, in accordance with aspects of the present disclosure; and

FIG. 16 depicts a graphical representation of comparison between an expected and observed depressor cage position in the event of a soft occlusion, in accordance with aspects of the present disclosure.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The syringe pump discussed herein can be used as a general purpose automatic syringe pump. In certain embodiments, discussed herein the automatic syringe pump also has the ability to store a variety of drug protocols via a microcontroller built into or in communication with the device. Such drug protocols may be for varied applications such as delivering cardiovascular or chemotherapy drugs. Also, the disclosed automatic syringe pump fills the need of a syringe pump that can provide reliable intravenous drug or fluid delivery over a period of 24 hours.

As discussed herein, the disclosed automatic syringe pump utilizes a ratchet and two pawl system to control the release of a spring's potential energy. Use of such a system allows regulated syringe depression, unlike traditional ratchet and pawl designs. In particular, the presently disclosed ratchet and two pawl system operates such that that, when one pawl is disengaged, the other pawl is engaged with the ratchet, thereby allowing the ratchet to rotate half a tooth and regulating depression of the syringe. As discussed herein, the present automatic syringe pump is electrically power efficient compared to conventional systems and can implement a variety of drug protocols. Furthermore, the separation of the power and regulatory components provides additional benefits. For example, in one embodiment in the case of an electrical malfunction, the pawls would prevent unregulated rotation. Likewise, in one implementation, in the case of pawl malfunction depression rate would be limited by the 2.32 lb driving spring force.

As discussed herein, the automatic syringe pump may have an upper and lower limit for the flow rates of the controlled syringe. The lower limit may be extended by utilizing a ratchet with more teeth, and the upper limit may be extended by adding a stronger constant force spring (CFS) to depress the syringe. Additionally, calibration may be performed for different syringe sizes for which the pump is to be used.

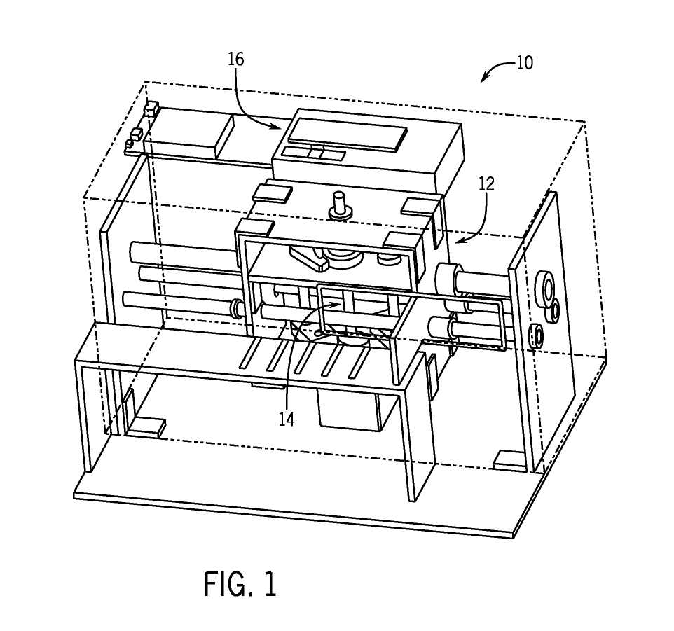

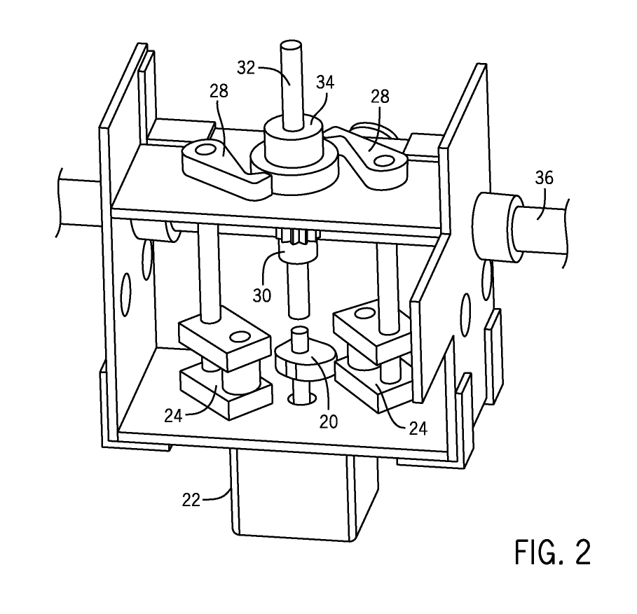

By way of general overview, FIG. 1 depicts an example of an embodiment of an automatic syringe pump 10 in accordance with the present disclosure having a power system 12, timing system 14, and user interface 16. An example of the timing system 14 is depicted in FIG. 2, which, among other features depicts a cam 20 attached to a stepper motor 22, wherein the cam 20 rotates to interact with flippers 24 of the pawl shaft to periodically disengage pawls 28 in an alternating fashion. Other features shown include pinion 30, drive shaft 32, ratchet 34, and round rack 36.

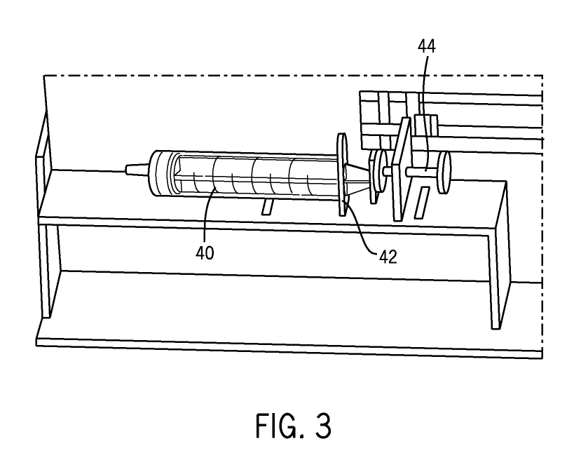

An example of the user interface 16 is depicted in FIG. 3, where a syringe 40 is depicted as being placed. The syringe 40 is placed in a syringe holder area configured to hold syringes of different sizes and that includes, in the depicted example, a series of spaced apart flange openings (e.g., slots) 42 as well as a threaded fine adjuster 44. Drug protocols for controlling actuation of the syringe 40 may be input by the user on a microcontroller, discussed below).

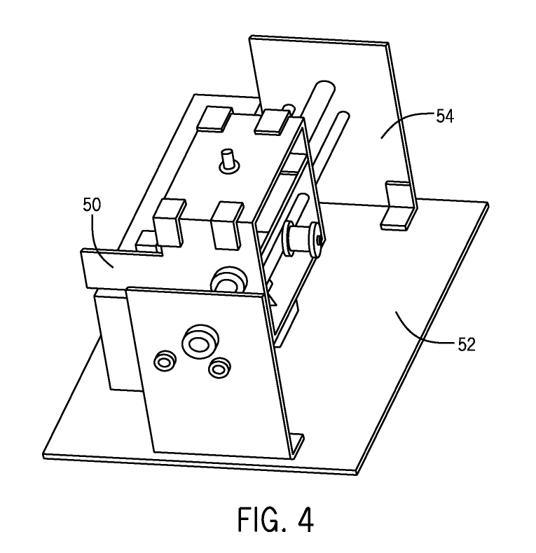

An example of a power system 12 is depicted in FIG. 4. In the depicted example, the power system includes a depression cage 50, spring holder 52, and spring mount 54. The mechanical arrangement in this example may be used to translate spring motion into depression of the syringe 40.

ASSEMBLY—In one implementation, the frame of the automatic syringe pump is made from aluminum or other suitable materials (e.g., acrylic, steel, brass), which would be machined into the parts dictated by the design plans, as discussed in greater detail below. In such an implementation, these machined parts would be connected to each other by brackets. Additionally, in such an implementation, the automatic syringe pump utilizes shafts that are cut to the length specified by the design drawings and which are placed in bushings which are placed into the machined parts. In addition, a constant force spring, pinion gear, pawls, and ratchet, are mounted onto the appropriate shafts.

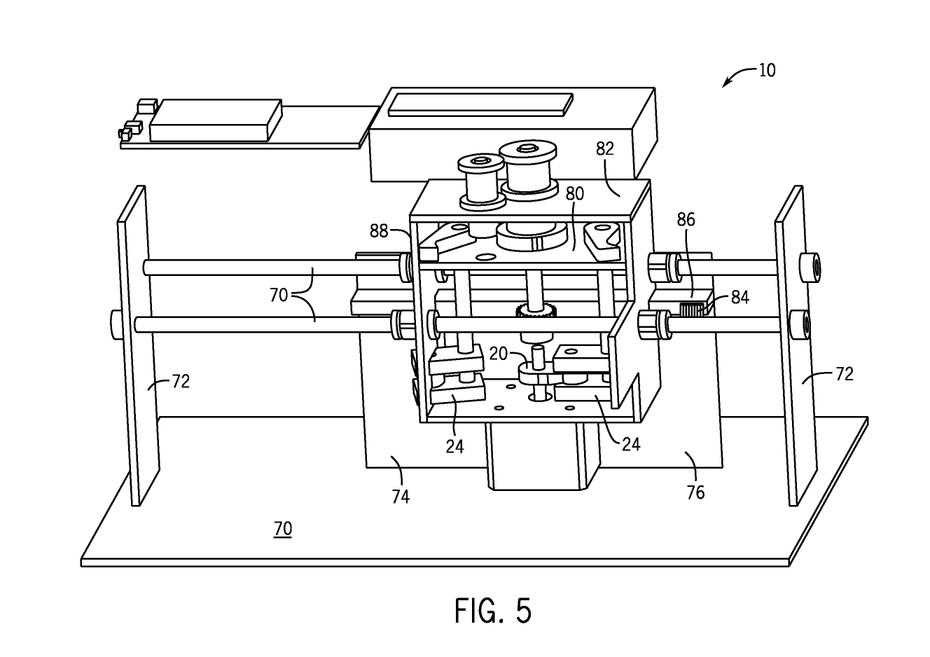

A more detailed description of how the pump 10 may be constructed is summarized below. As will be appreciated, in other embodiments, the pump 10 may be configured using different component parts or a different order of steps. With that in mind, and turning to FIG. 5, a schematic view of certain components of a pump 10 is shown to facilitate the present assembly discussion. In this depicted example, components include the cam 20, stepper motor 22, and flippers 24 already discussed. In addition, the example depicts a base plate 70, end plate 72, rack wall 74, depressing wall 76, motor wall 78, ratchet wall 80, constant force spring (CFS) wall 82, rack 84, rack standoff 86, linear sliding wall 88, and sliding shafts 90.

With respect to one implementation of assembly, the following general machining procedures may be employed where appropriate. Drilling—to reduce bit wobble, holes are generally drilled first with an undersized center drill, then with a bit that is 1/64 under gauge, then with the proper sized bit. Tapped holes are generally drilled with the proper, listed drill, then tapped using a tapered tap, then a full gauge tap. Milling—initially, designate the origin at the corner of each piece using an edge finder. In one implementation, trim 5 thousandths off of each edge to ensure flat edges. Measure the newly flattened edges, then use an end mill to cut to proper outside dimensions. Use the same drilling procedure for any holes drilled with the mill.

Base plate 70. Tools—Shear Press, Drill Press, File, 6-32 Taps, and Tap Wrench. Instructions—(1) Place an aluminum sheet in the shear press so that 12 inches is the width dimension. Cut at 8 inches as specified. (2) Mark the centers of all holes that will be used for corner brackets. Use a metal point to indent the centers, and drill the holes. (3) Tap the drilled holes to produce threads. (4) Add 4 rubber feet to the bottom of the base plate 70. (5) File the edges to remove any burrs.

End plates 72. Tools—Shear Press, Bridgeport Milling Machine, File, 6-32 Taps, and Tap Wrench. Instructions—(1) Place an aluminum sheet in the shear press and cut 2 rectangles according to the specified outside dimensions. Oversize the dimensions by ¼″. (2) Place both rectangles stacked on top of each other into the milling machine. Use the edgefinder to set the origin at a corner of the plates. (3) Use an end mill and digital caliper to cut down the outside dimensions of the plates to the exact size. (4) Mark the centers of all holes that will be used for corner brackets. Use a metal point to indent the centers, and drill the holes. (5) Tap the drilled holes to produce threads. (6) File the edges to remove any burrs.

Rack wall 74. Tools—Shear Press, Drill Press, File, 6-32 & 4-40 Taps, and Tap Wrench. Instructions—(1) Place an aluminum sheet in the shear press and cut a rectangle according to the specified outside dimensions. Do not oversize these dimensions—this piece will be too large to cut to exact dimensions on the mill. (2) Mark the centers of all holes that will be used for corner brackets. Use a metal point to indent the centers, and drill the holes. (3) Tap the drilled holes to produce threads. (4) File the edges to remove any burrs.

Rack standoff 86. Tools—Band Saw, Bridgeport Milling Machine, File, 4-40 Taps, and Tap Wrench. Instructions—(1) Cut a 6.25″ length of aluminum from a specified part using the band saw. Use an end mill to trim to the exact dimension. (2) Mark the centers of all holes that will be used for corner brackets. Use a metal point to indent the centers, and drill the holes. (3) Tap the drilled holes to produce threads. (4) File the edges to remove any burrs.

Rack 84. Tools—Hack Saw, Bridgeport Milling Machine, File, 4-40 Taps, and Tap Wrench. Instructions—(1) Cut a 6″ length of aluminum from a specified part using a hack saw. (2) Mill off the last three teeth on either end of the rack to produce a flat surface. (3) Drill a size 4-40 clearance hole into each newly flat end of the rack. (4) File the edges to remove any burrs.

Motor wall 78, CFS wall 82, ratchet wall 80. Tools—Shear Press, Bridgeport Milling Machine, File, 6-32 Taps, and Tap Wrench. Instructions—(1) Place an aluminum sheet in the shear press and cut 3 rectangles according to the specified outside dimensions. Oversize the dimensions by ¼″. (2) Place all 3 rectangles stacked on top of each other into the milling machine. Use the edge finder to set the origin at a corner of the plates. (3) Use an end mill and digital caliper to cut down the outside dimensions of the plates to the exact size. (4) Drill the center drive shaft hole through all three plates. (5) Remove one plate and then drill the pawl shaft holes through the remaining plates. (6) Remove one more plate and drill the four motor attachment holes. This is the motor wall 78. Go back and drill the remaining holes on the ratchet wall 80 and CFS wall 82 individually. (7) Tap the smaller diameter holes to produce threads. (8) File the edges to remove and burrs.

Depressing wall 76. Tools—Shear Press, Bridgeport Milling Machine, File, 6-32 Taps, and Tap Wrench. Instructions—(1) Place an aluminum sheet in the shear press and cut a rectangle according to the specified outside dimensions. Oversize the dimensions by ¼″. (2) Place the rectangle into the milling machine. Use the edge finder to set the origin at a corner of the plates. (3) Use an end mill and digital caliper to cut down the outside dimensions of the plates to the exact size. (4) Flip the plate vertically in the chuck to cut the necessary slots. (5) Wait until the linear sliding wall has been built to drill the holes. (6) File the edges to remove any burrs.

Linear sliding wall 88. Tools—Shear Press, Bridgeport Milling Machine, File, 6-32 Taps, and Tap Wrench. Instructions—(1) Place an aluminum sheet in the shear press and cut a rectangle according to the specified outside dimensions. Oversize the dimensions by ¼″. (2) Place the rectangle into the milling machine. Use the edge finder to set the origin at a corner of the plates. (3) Use an end mill and digital caliper to cut down the outside dimensions of the plates to the exact size. (4) Stack the dimensioned plate on top of the depressing wall 76 plate, line up, and drill holes. (5) Tap smaller diameter holes to prepare for corner brackets. (6) File the edges to remove any burrs.

Flippers 24. Tools—Band saw, Bridgeport Milling Machine, File, 6-32 Taps, and Tap Wrench. Instructions—(1) Cut four 1.25″ lengths from the rack standoff aluminum stock. (2) Use the end mill to cut these to exact rectangular dimensions. (3) Stack the four dimensioned plates on top of each other, line up, and drill holes. (4) Use the drill press to drill set screw holes in each flipper. Tap these holes. (5) File the edges to remove any burrs. (6). Cut two short flipper shafts, and attach the plates to the shaft using a set screw. The plates should be separated by a shaft collar. (7) The two flippers are now complete.

Cam 20. Tools—Band saw, Bridgeport Milling Machine, File, 6-32 Taps, and Tap Wrench, Belt Sander. Instructions—(1) Cut a 1.25″ length from the rack standoff aluminum stock. (2) Use the end mill to cut these to exact rectangular dimensions. (3) Drill a 5 mm motor hole as specified. (4) Use the drill press to drill a set screw hole and tap this hole. (5) Use the belt sander to round the edges of the cam 20.

Constant force spring (CFS) holders 52. Tools—Monarch Lathe, Bridgeport Milling Machine, File, 6-32 Taps, and Tap Wrench. Instructions—(1) Use the Monarch Lathe to turn specified cylindrical pieces. (2) Remove these parts and use the mill to produce CFS attachment holes. (3) Tap these holes. (4) Use a file to smooth edges.

Constant force spring (CFS) holders 52. Tools—Drill press. Instructions—(1) Use the drill press to ensure that both end of the constant force spring have holes that are compatible with a 4-40 screw.

Sliding shafts 90. Tools—Grinder. Instructions—(1) Use the grinder to cut two 12″ lengths of hardened shaft. These will be the linear sliding shafts 90. (2) Use the grinder to chamfer the cut edges.

Pawl shafts, drive shaft 32. Tools—Hack saw. Instructions—(1) Use a hack saw to cut the two pawl shafts and the drive shaft as specified. (2) File the cut edges.

Once all parts have been machined, assemble the pieces as follows: (1) Attach the two end plates 72 to the base plate 70 using corner brackets and screws. (2) Attach the rack wall 74 to the base plate 70 using sliding corner brackets and screws. (3) Attach the rack standoff 86 to the rack wall 74 using 90 degree aluminum and screws. (4) Attach the rack 84 to the rack standoff 86 using two screws. (5) Assemble the timing system walls (e.g., motor wall 78, ratchet wall 80, CFS wall 82, sliding wall 88, depressing wall 76) using corner brackets and screws. (6) Attach the stepper motor 22 to the motor plate 78 using screws. (7) Press rotational bearings into all pawl shaft holes and the two drive shaft holes. (8) Insert the pawl shaft through the bearings and slide on: the flippers 24, the pawls 28, the torsional springs. Lock the shafts in place using collars. (9) Insert the drive shaft 32 and attach the pinion gear 30 and ratchet 34 using the set screws. Lock in place using a collar. (10) Attach the constant force spring to the CFS holder spools using screws. Attach the CFS holder large spool to the drive shaft 32 using a set screw. Slide the small spool onto a small spool shaft and lock in with a collar. (11) Place linear bearings into the four sliding holes and lock in place with retaining rings. Place the sliding shafts 90 into the end plate 72 and slide through the linear bearing on the timing assembly 14. Lock into place using collars. (12) Set up the syringe holder device using corner brackets.

DESIGN EXAMPLE—While the preceding gives a generalized overview of certain aspects of the automatic syringe pump presently contemplated, as well as its assembly, the following provides a more detailed discussion of a specific example of such a device.

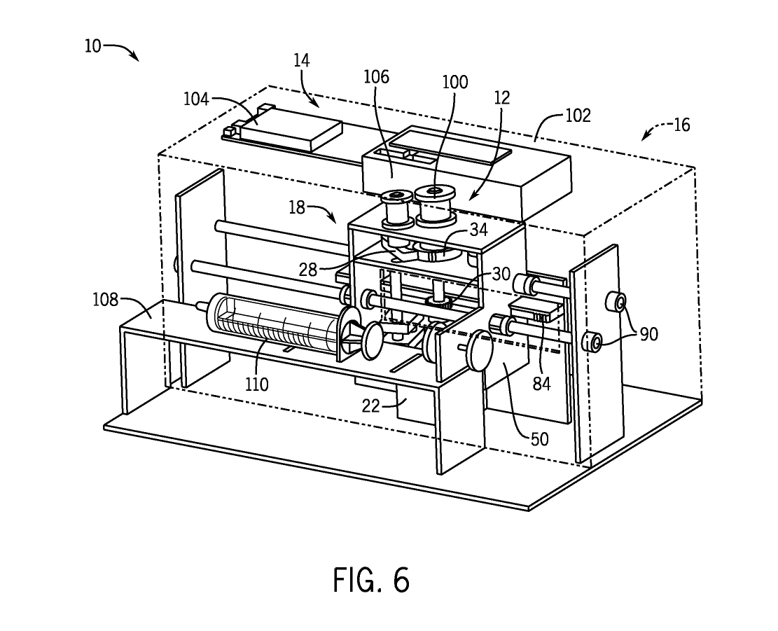

As noted above, the present automatic syringe pump is designed to have a combination of mechanical components that provide the power for syringe depression and electronic regulation that would allow for a wide range of variability and control. The automatic syringe pump 10 depicted in FIG. 6, can be broken down into four main subsystems: the mechanical power supply 12 (including constant force spring 100), the electronic timing system 14 (including microcontroller 102, ratchet 34 and pawls 28, and stepper motor 22), the mechanical power transfer system 18 (including rails 90, rack 84, pinion 30, and depressor cage 50), and the user interface 16 (including Arduino backpack 104, LCD display/microcontroller 106, and syringe tray 108). Though the constant force spring 100 is depicted in Certain of the figures as being disposed on the top of the pump 10, it should be appreciated that in other embodiments, the spring 100 may be situated at other locations of the pump 10, such as one the sides of the pump 10 or at any other location suitable for applying compressive force to a driveshaft 32. In the depicted embodiment, a constant force spring-powered driveshaft 32 causes rotation of a pinion gear 30 that moves along a rack 84. As the pinion gear 30 moves along the rack 84, the depressor wall 76 on the depressor cage 50 depresses a syringe 110. The rate of driveshaft rotation is regulated through the electronic timing subsystem 14. These subsystems will be described in detail to provide a better understanding of each component.

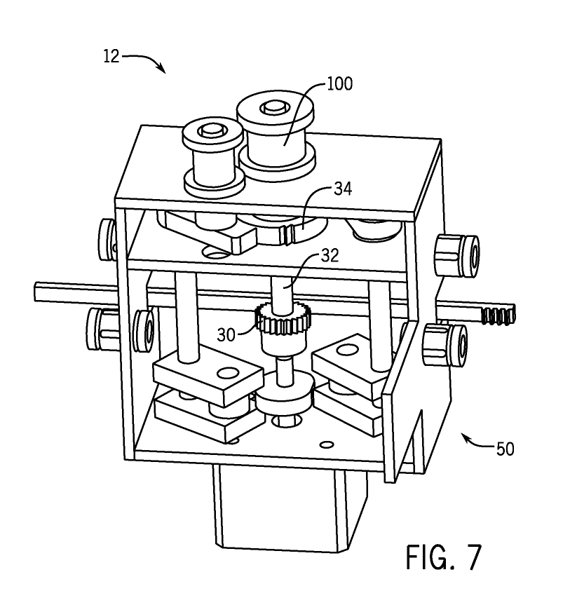

With respect to the subsystems, FIG. 7 depicts an example of a mechanical power subsystem 12 in conjunction with a simplified view of a depressor cage 50. The mechanical power subsystem 12 uses a constant force spring 100 which sits on top of the depressor and is mounted on the driveshaft 32 seen in the figure. This spring 100 holds potential energy and tries to transfer that energy through the driveshaft 32 to cause rotation of the ratchet 34 and pinion gear 30.

As seen in the example depicted in FIG. 7, the constant force spring 100 sits on top the sliding case. The constant force spring 100 is wrapped around two drums. The main drum is set screwed onto the drive shaft 32 and is the drum that allows the spring 100 to impart torque to the drive shaft 32. The second drum sits off to the side of the main drum, but instead of being set screwed onto the shaft 32 this drum just rotates around the shaft 32 as needed to ensure that no twisting occurs between the main drum and the second drum. If nothing is done to mediate the release of the energy stored by the constant force spring 100 the pump 10 would be limited in flow rate variability. To address this issue, an electronically controlled timing system that regulates the release of the energy stored in the spring 100 is employed.

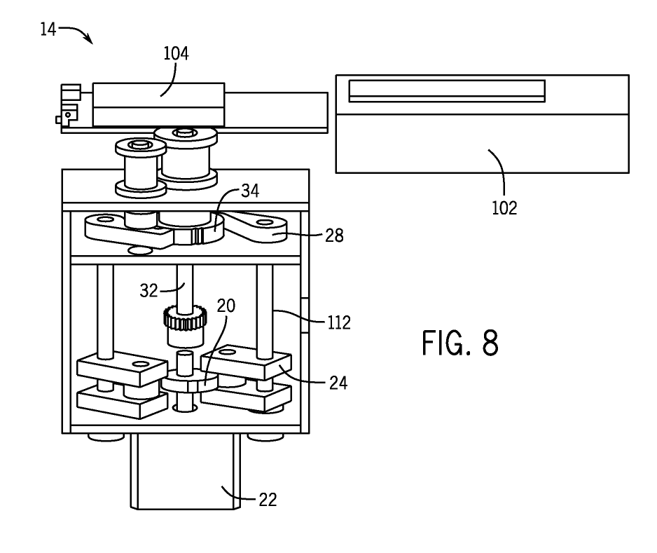

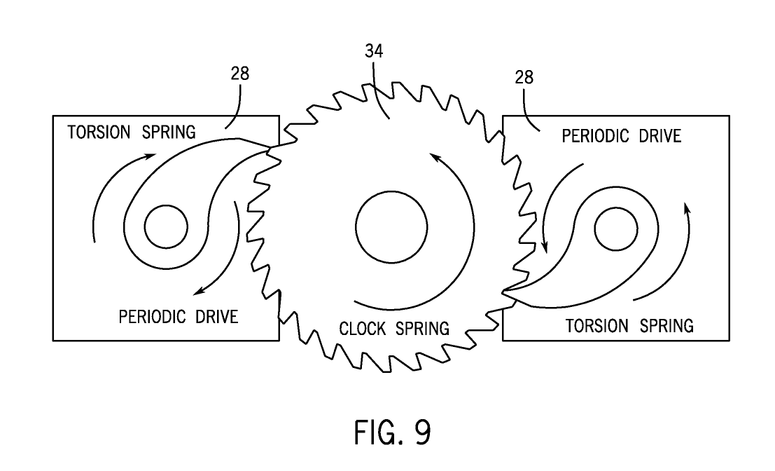

In particular, an example of a suitable electronic timing system 14 is depicted in FIGS. 8 and 9. As depicted in FIG. 8, and as discussed below, the depicted timing subsystem 14 is composed of the ratchet 34, two pawls 28, two flippers 24, cam 20, stepper motor 22, microcontroller 102 (such as a low-power microcontroller), and Arduino backpack 104. The depicted timing subsystem 14 uses the above components to resist the rotation of the drive shaft 32. The drive shaft 32 is allowed to rotate when the stepper motor 22 causes one pawl 28 to disengage. This arrangement is depicted in FIG. 9, which shows a focused view of 2-pawl electronic escapement. In this example, both pawls 28 are pressed against the ratchet 34 due to torsional springs on the flippers 24. But due to the positioning of the pawls 28, only one pawl 28 is engaged with a ratchet tooth at an instant. The pawls 28 are disengaged from the ratchet 34 by the rotation of one stepper motor 22 with a cam 20.

With the foregoing in mind, the focus of the electronic timing system 14 as previously stated, is to regulate the release of the energy stored in the constant force spring 100. The spring 100 places a torque on the driveshaft 32. However, the drive shaft 32 is not allowed to rotate because one pawl 28 is engaged with the ratchet 34. The other pawl 28, as discussed with respect to FIG. 9, is in between two teeth of the ratchet 34 and is not bearing any load. To control the release of the stored energy, the stepper motor 22 with cam 20 is used to cause the pawls 28 to disengage with the ratchet 34. As seen in FIG. 8, each pawl 28 is connected to a flipper 24 via a shaft 112. The flippers 24 are placed in the depicted example so as to reduce the number of stepper motors 22 from two to one. The stepper motor 22 is attached to a cam 20. As the cam 20 rotates, it comes in contact with one of the flippers 24. When the cam 20 comes into contact with the flipper 24, it causes the flipper 24 to deflect. This deflection is transmitted down the shaft 112 and causes disengagement of the pawl 28 from the ratchet 34. Once the pawl 28 is disengaged, the ratchet 34 is allowed to rotate only half a tooth because the ratchet 34 then becomes engaged with the other pawl 28. To ensure the pawls 28 are never both disengaged from the ratchet 34, the cam 20 is configured so that it can only be engaged with one flipper 24 at a time. For example, this may be accomplished by making one end of the cam 20 longer than the other end such that when the long end rotates around it will contact the flipper 24.

The microcontroller 102 regulates how fast the stepper motor 22 and cam 20 rotate, and therefore dictates the flow rate of the pump 10. In one embodiment, the microcontroller 102 is encoded to run both the stepper motor 22 and an LCD display 106. An example of code to be executed on the microcontroller 102 is reproduced herein. The example of code reproduced herein creates a menu that may be navigated by the user. This menu takes inputs that allow the user to specify a certain flow rate and syringe size or specify a pre-programmed drug protocol. The microcontroller 102 then calibrates for the user's input and determines the rate at which the stepper motor 22 must rotate to achieve the user's specified flow rate. When the user hits start, the microcontroller 102 begins to rotate the stepper motor 22 and syringe depression begins. The last portion of the electronic timing system 14 is composed of the Arduino backpack 104. This component is discussed below in the context of the user interface subsystem 16.

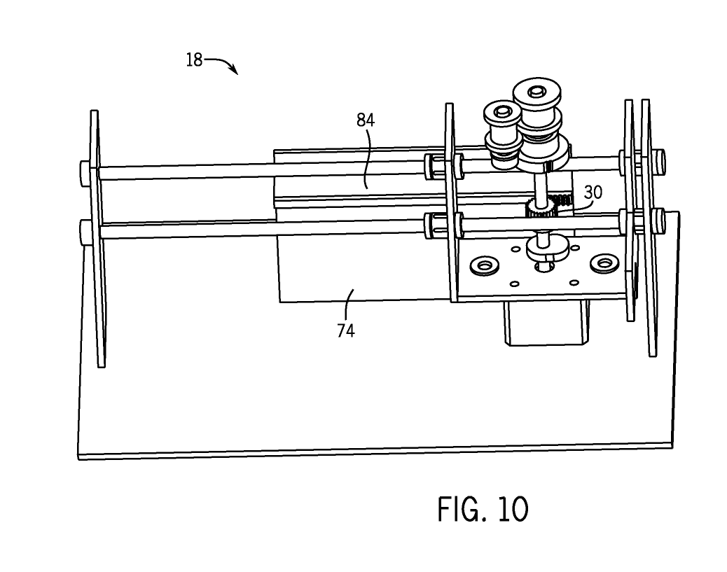

We will now examine how the spring's stored energy is used to create motion and depression of the syringe 110. Turning to FIG. 10, a simplified view of mechanical power transfer subsystem 18 is depicted. In this example, one component of this subsystem 18 is the rack 84 and pinion 30. The remainder of the mechanical components help ensure alignment and support the rest of the device. The rack 84 and pinion gear 30 allow for the translation of rotational motion to linear motion.

In particular, the mechanical power transfer subsystem 18 reveals how the automatic syringe pump 10 utilizes the rotational motion generated by the constant force spring 100 to result in the depressing of a syringe 110. The rack 84 and pinion 30 system couples rotation of the driveshaft 32 to the horizontal displacement of the depressor. The depressor has a wall 76 attached to it that is used to depress the syringe 110. Thus, when the depressor moves along the rails 90, the depressor wall 76 also moves and depresses the syringe 110. The depressor has four linear bearings that allow for smooth motion along the rails 90 so that there is minimal resistance to the movement of the pinion 30 on the rack 84. The last component of this subsystem 18 to be discussed is the rack plate 74. This component is used to get the rack 84 to the correct position to provide the optimal meshing of teeth between the pinion 30 and rack 84. The rack 84 may be secured with adjustable brackets such that it can be moved closer or further from the pinion 30 to ensure optimal meshing.

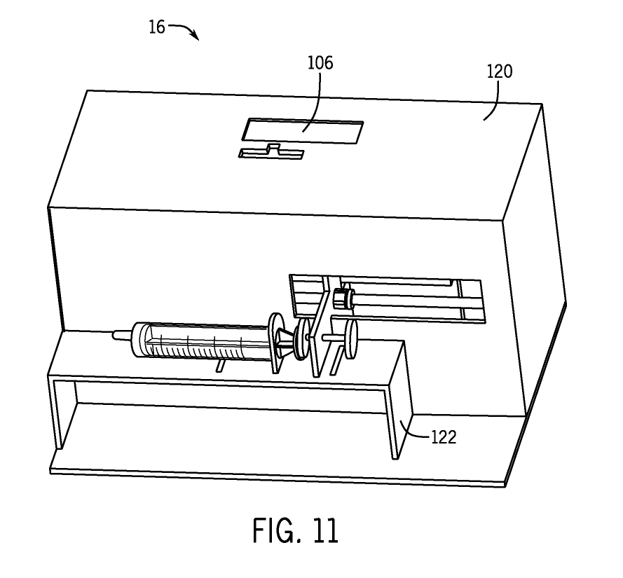

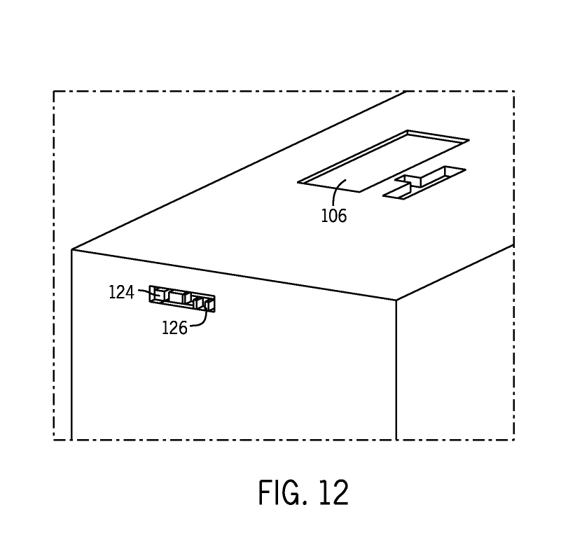

To perform as an automatic syringe pump that can be readily used, the device 10 also includes a user interface 16 that makes it easy for the user to utilize the automatic syringe pump 10. Turning to FIGS. 11 and 12, an example of a suitable user interface 16 is depicted and discussed. Turning to FIG. 11, a front view of a simplified user interface subsystem 16 is depicted. Two of the main components of this subsystem 16 are shown in this figure: the syringe tray 122 and LCD display 106 attached to the microcontroller 102. Turning to FIG. 12, a side view of the user interface 16 is depicted. In this example, the Arduino backpack 104 is depicted. The majority of the backpack 104 is contained inside of the lid 120, and the remaining portion of the backpack 104 is accessible to the user. In this example, the backpack 104 has an accessible connector port 124 (e.g., a USB port) for power input and a switch 126 that changes the backpack 104 between the charging, off state, or the powered state. In one embodiment, the microcontroller 102 and Arduino backpack 104 used in the pump 10 are enclosed and mounted onto an aluminum casing with standing screws which helps shield the microcontroller 102 and Arduino backpack 104 from damage.

In operation, the LCD display 106 and microcontroller 102 may be used to display user options and receive user inputs. For example, a home screen may be displayed on the LCD 106 after powering on the automatic syringe pump 10. Pressing the right key (or other input structure) will allow the user to navigate through other protocols. Similarly, the LCD 106 may be used to display to the user the selections that can be made so that have been made when working under manual operation. For example, the LCD 106 may be used to display the selected syringe size and flow rate. The units may be displayed on a separate, preceding screen if space is limited to ensure the correct syringe and flow rate are chosen.

With the foregoing in mind, and to elaborate on the use of one embodiment of the user interface the user will interact with the automatic syringe pump in three main ways: placing the syringe 110 in the device, setting the microcontroller 102 for a desired setting, and turning on and charging the device 10. As seen in FIG. 11, the syringe tray 122 may be used to bring a syringe 110 to the necessary height to be engaged by the depressing wall 76. This tray 122 may have a reversible zip-tie attached to it (or other securing mechanism), which may be used to secure the syringe 110 in place. In one embodiment, the microcontroller 102 receives input via five buttons that the user will be able to use. One button will be designated as the start button while the other buttons (e.g., up, down, right, and left) will be used to control movement through the menus of the microcontroller 102 displayed on the LCD 106, allowing options like syringe size and flow rate to be specified. The microcontroller 102 can also have programmed protocols which will allow the user to quickly give a drug or fluid at a previously specified rate. In addition to setting the automatic syringe pump 10 to depress a syringe 110 and placing the syringe 110 on the syringe tray 122, the user may also power on/off the device 10 and charge the device 10. As seen in FIG. 12, these components may be placed on the side of the device 10 (e.g., connector port 124 and power switch 126). In the depicted example, the USB plug can be constantly plugged into the device 10 and may be used to operate the device while power is present. When power is not present, the device may run off of a rechargeable battery, such as may be present in the Arduino backpack 104. In one embodiment, the backpack 104 cannot be charged and supply power to our device at the same time, and so to charge, a switch on the side of the device may be switched to the charge position. Lastly, this subsystem 16 may also incorporate the ability of the user to easily interact with the device 10, allowing the device to be built in relatively small size factors, such as a size of 8″×12″×6″. Overall, the user interface 16 is designed to be self explanatory such that the user will not need significant training to operate the device 10.

With regard to the design criteria associated with the automatic syringe pump 10, in certain embodiments, criteria may be considered such as: (1) Accuracy—The device will be administering fluids or medication to patients at precise rates. It may be desirable for flow rates to be accurate within +/−5%. (2) Flow Rate Variability—The device 10 may accommodate the largest amount of specified treatments to ensure it is usable. Additionally, the device 10 may accommodate treatments that require different flow rates at different times for the same patient. For example, many thrombolytic drugs have complicated equations to determine flow rate as a function of time. It may be desirable for flow rates to be adjustable in 0.25 cc/min increments. Therefore, it may be desirable for the device 10 to be able to adjust flow rate for drug delivery regimen without human intervention after setup. It may be desirable for volume flow rates to be accurate within the range of 5 to 90 mL/hr. (3) Ease of Operation/Usability—The device may be easily and timely set-up. It may, therefore, be desirable for the device to be able to be set up in less than 1 minute per operation, run all drug flow regimens without any medical staff intervention, and should score greater than 80 on Systems Usability Scale. (4) Portability—It may be desirable for the device 10 to be portable. For example, the device 10 may weigh less than approximately 16 lbs and may have a carrying handle. (5) Compatibility—The device 10 may be compatible with the syringes that are in common usage throughout the world to ensure maximum usability. For example, it may be desirable that the device 10 be able to accommodate BD SmL, 10 mL, 20 mL, 40 mL and 60 mL syringes as well as any syringes with small variations in dimensions. (6) Size—Clinics may be cramped and/or small. Therefore, it may be desirable for the device to occupy a limited footprint, such as a footprint no larger than 11″×12.5″×12″. (7) Energy Consumption—It may be desirable that energy consumption not drain the total charge stored in the battery before 24 continuous hours, thus allowing our device 10 to operate for 24 continuous hours. In an embodiment where a battery of the device holds a total charge of 2400 mAh, the energy consumption of the device 10 would be less than 100 mA to ensure the device operates for 24 continuous hours. Thus, it may be desirable for the energy consumption of the device 10 to be less than 100 mA. In one embodiment, the Arduino backpack 104 contains 2400 mAh of charge. From preliminary measurements, the microcontroller 102 supplies the stepper motor 22 with 2.38 mA, which gives a theoretical lifetime of 1000 hours of operation before the battery would run out.

With respect to these design criteria, the accuracy of the presently disclosed pump 10 is controlled by each subsystem described above. Accuracy is achieved by providing a reliable and calculated release of fluid on each half tooth rotation of the ratchet 34 which results in certain depression of the syringe 110. In one embodiment, the pump 10 is calibrated for a certain number of syringes and syringe sizes so the user utilize syringes from the group that have been calibrated for the pump 10, and the user indicates the syringe size that they will use to ensure that the pump 10 provides accurate results. The mechanical power transfer subsystem 18 contributes to accuracy by ensuring that the rotation of the drive shaft 32 is translated into linear motion that depresses the syringe 110. The mechanical power subsystem 18 further ensures the accuracy of the pump 10 by providing a constant force that is strong enough to fully depress each syringe 110 calibrated for the pump 10. Finally, the electronic timing subsystem 14 also ensures that the pump 10 is accurate. In one embodiment, the microcontroller 102 in this subsystem executes code which is calibrated to produce a specific rate of motor rotation based on the type of syringe 110 (BD, Terumo, etc), the size of the syringe 110, and the rate of depression of the syringe 110. The microcontroller 102 uses this information to determine a specific rate of rotation of the stepper motor 22 and therefore the cam 20 on the stepper motor 22. As the cam 20 rotates, it deflects the flipper 24, which allows the ratchet 34 to rotate half a tooth. This rotation is transmitted through the driveshaft 32 to rotation in the pinion gear 30. The pump 10 is capable of providing accuracy over a wide range of flow rates because the half tooth rotation translates to a minute amount of linear motion. Since this amount of motion is so small, the syringe 110 can be depressed in small increments that allow an accurate dosage to be applied.

To facilitate usability of the automatic syringe pump 10, in certain embodiments the syringe tray 122 has a designed holder that suggests the correct position of syringe placement. Further, setting the pump 10 only involves pulling the depressing wall 76 to its initial position. In addition, in certain embodiments, the microcontroller 102 causes display of a menu that allows the user to scroll the options and select a desired drug or fluid delivery method. The menu includes arrows that correspond to the up, down, left, and right keys to make navigation through the menu self explanatory. Also, in certain embodiments, when the pump 10 is running, the microcontroller 102 provides the user with the length of the procedure as well as with the ability to pause the procedure in case of errors.

With respect to the automatic syringe pump 10 being usable with a variety of syringe sizes, the pump 10 satisfies this criterion mainly through the electronic timing subsystem 14. For example, in one implementation, the microcontroller 102 in this subsystem executes code which has the calibrated dimensions of each size of syringe 110 for which the pump 10 is calibrated. It then uses the calibrated dimensions to determine the rate at which the stepper motor 22 should spin to give an accurate amount of drug or fluid. Additionally, the pump 10 has the ability to be programmed with more syringe sizes, which allows for the possibility of increasing the device's compatibility. Also, the syringe tray 122, which is part of the user interface subsystem 16, may have a reversible zip-tie around it that can be used to secure syringes ranging from 5-60 mL.

With respect to the design criterion of flow rate variability, the main subsystem used to satisfy this design criterion was the electronic timing subsystem 14. The microcontroller 102 in this subsystem controls the rate of syringe depression based on the rate of rotation of the stepper motor 22. After receiving the commands or protocols from the user, the microcontroller 102 calculates the rate at which the stepper motor 22 needs to spin to give the desired dose. The ability of the microcontroller 102 to vary the rate of the stepper motor 22 shaft rotation corresponds to the ability of the pump 10 to vary flow rates. That is, as the stepper motor shaft rotates the cam 20 attached to shaft results in the deflection of the flipper 24 and pawl 28 as a result. As the pawl 28 is disengaged from the ratchet 34, the ratchet 34 rotates half a tooth and this rotation is transmitted to the pinion gear 30 and through the rack 84 is translated into linear motion. This linear motion is connected to the depression of the syringe 110. If the rate of pawl disengage is increased, which would occur when the rate of the stepper motor shaft is increased, there would be more rotation of the ratchet 34 and pinion 30 accordingly in the same amount of time. This increased amount of rotation would correspond to an increased amount of linear motion, which means that in the same amount of time the syringe 110 would have been depressed more. Thus, the automatic syringe pump 10 is able to provide a wide range of flow rates due to the ability of the pump 10 to change the speed at which the pawls 28 are disengaged from the ratchet 34 via the cam 20 on the stepper motor shaft and via operation of the microcontroller 102.

To minimize the size of the pump 10, in one embodiment the rack 84 and pinion 30 mechanism may be implemented in a way that is not conventional. In particular, instead of the rack 84 moving across the pinion 30, the pinion 30 moves across the rack 84. Such an implementation reduces the overall size of the pump 10.

In conclusion, in certain embodiments, the automatic syringe pump 10 is composed of four subsystems: mechanical power 12, electronic timing 14, mechanical power transfer 18, and user interface subsystems 16. The mechanical power subsystem's main component is a constant force spring 100, which is capable of storing the energy needed to reliable depressed several different sizes of a syringe 110. The electronic timing subsystem's major components are the ratchet 34, pawls 28, flippers 24, stepper motor 22, and microcontroller 102. These components are used together to regulated the release of the energy stored in the constant force spring 100. The mechanical power transfer subsystem's main components are the rack 84 and pinion 30 which translates the rotational motion of the drive shaft 32 from the constant force spring 100 into linear motion to depress the syringe 110. Finally, the user interface subsystem's main components are the microcontroller 102, Arduino backpack 104, and syringe tray 122.

As will be appreciated, there are several components of the automatic syringe pump that can be varied. For instance, in other embodiments, a different approach could be used to regulate the energy release of the spring 100 such as using a balance wheel or using pins that would prevent the depression of a spring. Additionally, a different source of energy could be used to power the entire device so the device could be gravity powered, electrically powered, etc. In addition, while a square rack 84 is depicted in certain of the figures as discussed herein, it should be appreciated that in other embodiments the tack 84 may be of other shapes or geometries, such as a round rack. Further, in other embodiments, the constant force spring 100 may be wound, such as using a knob, instead of being wound when the depressing wall 76 is moved from the full depressed position to the beginning position. Further, though present embodiments have been described in which the rack 84 and pinion 30 are not movable so that the pinion gear 30 will always stay in contact with the teeth of the rack 84, in other embodiments the rack 84 and pinion 30 may be movable.

While the foregoing describes one embodiment of an automatic syringe pump, other embodiments may be employed, such as embodiments that detect pressure occlusions. Occlusions may be generally be characterized as being hard (i.e., complete) occlusions or as being soft (i.e., partial) occlusions. Hard occlusions are characterized by the complete obstruction of fluid flow. Typically it is a mechanical obstruction arising from closed stopcocks, kinked tubing/catheters, etc. Soft, or partial, occlusions result instead in a reduction of fluid flow, not a complete stoppage, still permitting some amount of fluid to move. Soft occlusions may occur due to a build up of precipitates in the catheter, compression of the catheter, pressing of the catheter against a vessel wall, or the penetration of the catheter into tissue space outside vessel.

Occlusions may interrupt the flow of medication to the patient, effectively stopping or slowing treatment. The increase in pressure possibly resulting from occlusions is typically not believed to be a threat to the patient. In particular, because the venous system possesses very high compliance, it is unlikely that a pump (IV, infusion, syringe, etc.) would be capable of raising the pressure of the venous/arterial system. On the other hand, the injection of a bolus upon release of the occlusion may be undesirable with respect to the prescribed treatment regime of the patient.

In certain embodiments a pressure occlusion detection device may be incorporated that will detect pressure increases, such as to sound an alarm and cut off the medicine to the patient. For example, a device may be employed that detects pressure increases within IV lines and releases the pressure with a mechanical relief valve while setting off an alarm to alert the heath care providers of the pressure occlusion. In one such embodiment, attached to the IV line is a t-valve that would be connected to a pressure relief valve. If pressure inside the IV line exceeded a certain pre-set amount, the valve would open, and medication would flow out of the pressure relief into an auxiliary line leading to an auxiliary area. The device would only require two AA batteries to run with the alarm. When the medication (ionic) leaks out of the relief valve, a circuit will be complete so that the alarm will sound.

For example, in one implementation a t-valve is attached to the IV line of the automatic syringe pump. Attached to the t-valve is a pressure relief valve. On the other side of the pressure relief valve is an auxiliary line leading to an auxiliary reservoir. When pressure exceeds a certain pre-set amount in the IV line, the pressure relief valve opens and diverts medication away to the auxiliary line to release pressure.

Alternatively, in other embodiments, a potientiometer (e.g., a linear potentiometer such as the SoftPot potentiometer obtainable from SpectraSymbol) may be employed in place of a pressure analyzing system. In one such embodiment, an actuator moves with the depressor cage 50 along the potentiometer to indicate displacement. By basing the detection of occlusion on the displacement of the depressor cage 50 rather than a measured in-line pressure, there is no need to explicitly define an “occlusion pressure” since the detection is now directly associated with the flow of fluid.

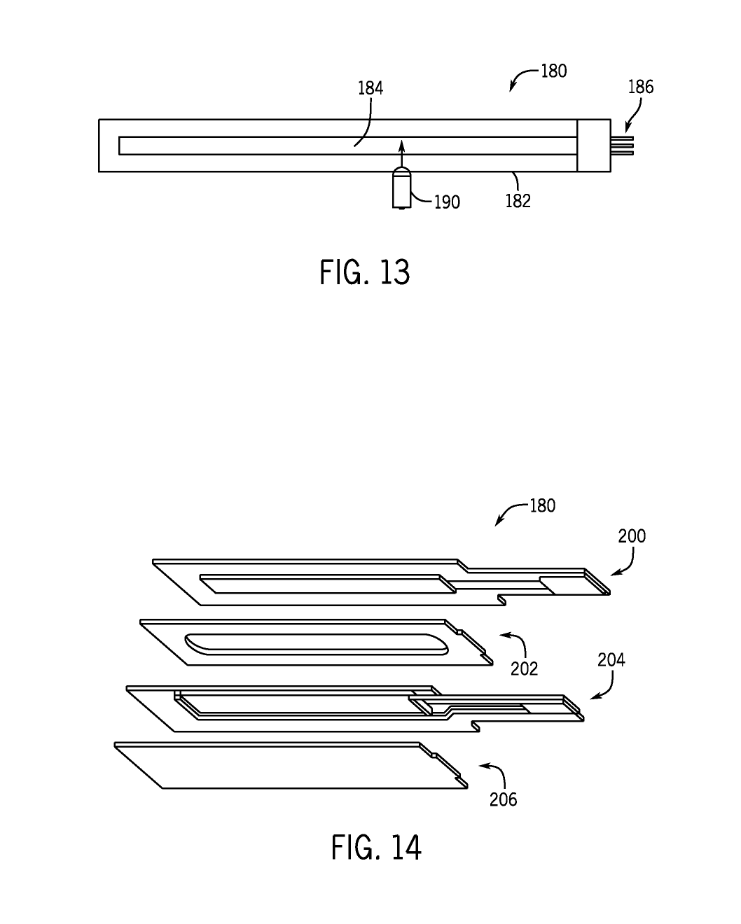

By way of example, FIG. 13 depicts an example of a rectangular potentiometer component 180 (having a body 182, sensing or sensitive area 184, and connector pins 186) along with an actuator 190. In one such implementation, the linear potentiometer 180 exhibits variable resistance that varies based on the position of the actuator 190 (which may be a separate piece) along the length of the linear potentiometer 180. In one implementation, the linear potentiometer is a rectangular circuit, such as a flexible circuit) that can be adhered to surfaces. As will be appreciated, though linear potentiometers are generally discussed herein, the potentiometer may be any suitable configuration, such as a circular configuration. In one embodiment, depicted in FIG. 14, the potentiometer 180 may be a layered construct, such as a construct having a top circuit or layer (e.g., a collector) 200, a circuit spacer 202, a bottom circuit or layer (e.g., a resistor) 204, and an adhesive layer 206 for securing the potentiometer 180.

In one example, the potentiometer 180 produces signals (i.e., is activated) in response to the actuator 190 pressing down on the sensing area 184, thereby causing the top circuit 200 and bottom circuit 204 to come into contact. When the top circuit 200 and bottom circuit 204 come into contact, an output is created that is dependent on the actuator's position along the sensing area 184, thereby generating a potentiomeric output. In one embodiment, the actuator 190 is designed to produce up to, but not exceeding, 3 N of force due to the action of an inner spring provided within the actuator 190, thereby preventing damage to the potentiometer 180 and reducing possible noise from variable amounts of pressure applied to the potentiometer 180.

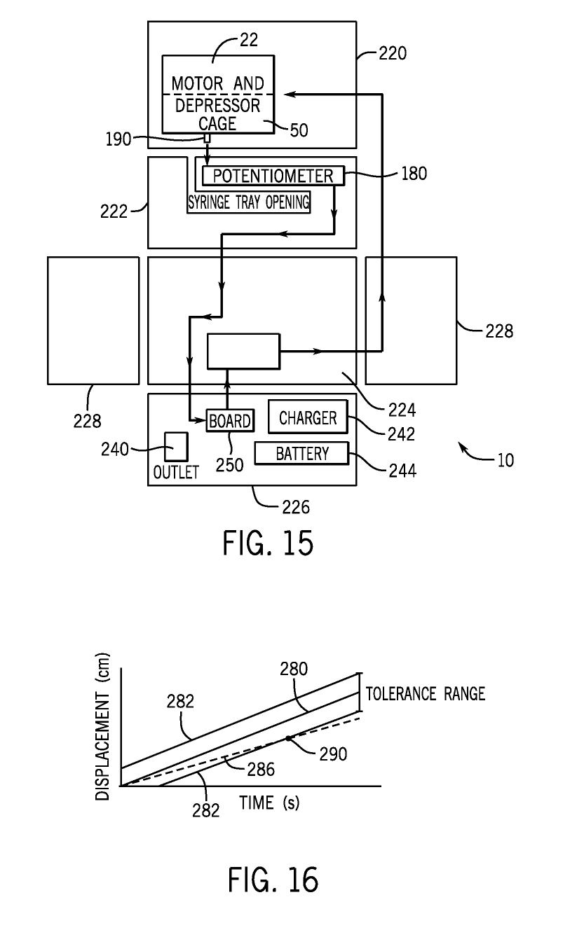

With the preceding in mind, FIG. 15 depicts an example of an implementation incorporating a potentiometer 180 as discussed herein. In particular, FIG. 15 depicts a control flow view of the operation of such a potentiometer in the context of an opened of unfolded automatic syringe pump 10. In this example a bottom plate 220, front plate 222, top plate 224, back plate 226, and side plates 228 are depicted along with their associated components. For example, bottom plate 220 is depicted along with the associated motor 22 and depressor cage 50, to which the actuator 190 is attached. The front plate 222 includes the syringe tray opening. The back plate 226 includes features such as an outlet 240, charger 242, and battery 244.

In this example, the actuator 190 contact the potentiometer 180 (e.g., a linear potentiometer) to generate an output signal that is communicated to a circuit board 250, which in turn (in this example), communicates with a microcontroller 102 (such as an Arduino device). In response to this input, control signals may be generated that control operation of the motor 22 and movement of the depressor cage 50.

With the preceding in mind, in one implementation the occlusion alarm mechanism functions as follows: at the beginning of each protocol, the microprocessor calculates a theoretical path by using the selected syringe size and flow rate. With respect to the theoretical path, it calculates the theoretical positions at which the depressor cage should be at each tick, based on the programmed flow rate. At the start of each tick, the microprocessor collects the position data of the depressor cage by means of the potentiometer. For example, in the depicted implementation, the depressor cage 50 has attached to it a wiper or actuator 190 that is constantly in contact with the sensing area 184 of the potentiometer 180. Through this setup the microprocessor is able to determine the position of the depressor cage 50 from the potentiometer signals. The measured position of the depressor cage 50 is then compared against the theoretical position, and if the position does not fall within a specified tolerance, an occlusion alarm is activated, stopping the device and, in one example, displaying on the screen an alarm message (e.g., “Occlusion Detected.”). An auditory signal or alarm may also be triggered in certain embodiments. An example of such a determination is shown in FIG. 16, in which a graphical representation is shown depicting the theoretical or expected position 280 of the depressor cage and the acceptable tolerance range 282 about the expected position 280.

With the preceding in mind, the potentiometer-based sensing mechanism is able to detect a partial or soft occlusion, when the partial or soft occlusion eventually causes a sufficient deviation (shown by line 286) from the predicted or expected position 280. At point 290 the deviation exceeds the specified threshold, triggering an alarm. In other words, since the partial occlusion reduces flow (rather than stopping it entirely), this reduction in flow results in a reduction in depressor cage displacement rate and eventual measurable deviation from the expected position. The system 10 (e.g., via the potentiometer or Arduino) records this reduction in displacement rate and will detect the deviation once it passes the tolerance threshold 282, as shown in FIG. 16.

As will be appreciated, though the preceding discussion pertains to partial occlusions. A full or complete occlusion will result in a complete stop in fluid flow. In one study, it was observed that the occlusion pressure, or the pressure at which the depressor cage 50 stops moving completely, was 147 mm Hg (200 cm H₂O). The testing procedure consisted of running protocols against a defined in-line pressure, generated by an effective water column by elevating the tubing. The height of the water column was incrementally increased by 10 cm (starting at a height of 40 cm) until no flow occurred. Fluid flow was determined by depression of the syringe itself, marking the start of the syringe depressor and inspecting if the black rubber band changed position. As the definition of a hard occlusion is the complete stoppage of flow, the determination of this pressure offers a physical value and condition under which a hard occlusion occurs.

As will be appreciated with respect to the potentiometer embodiment, although the occlusion detection is based on the displacement rate, it is still dependent on pressure. In other words, there is a specific in-line pressure (e.g., 147 mm Hg) at which the spring 100 will not be able to overcome the force and the depressor cage 50 will stop. However, the potentiometer-based approach allows detection of the moment when displacement stops, rather than attempting to define an occlusion pressure at which the cage 50 might stop. In essence, the occlusion is still dependent on pressure, but detection of the occlusion is not.

With respect to the tolerance 282 that may be specified, this tolerance may represent the value of the deviation at which automatic syringe pump will detect an occlusion. Since this is easily programmable, the tolerance, in one implementation, may be set to perform at the guaranteed accuracy determined by tests over the range of expected back-pressures (±100 mmHg according to the ISO standards). This further ensures that the accuracy will be at the guaranteed value. By testing and making sure that the correct volume is delivered within this range, an accuracy level will be able to be determined, which will then be translated to the tolerance of the occlusion alarm.

The above-described potentiometer approach offers a variety of advantages. First, this approach obviates the need to calibrate or integrate a pressure sensor by instead measuring a physical property of the device (i.e., displacement) that may indicate an occlusion. Further, this approach can respond to other malfunctions that may stop the dispensing of the medication (by stopping the depressor cage 50). In addition, this approach offers easy implementation without needing to place anything in contact with the medication, while minimizing the addition of further electronic components, thereby lowering cost and power consumption. By continuously calculating the expected displacement, the potentiometer-based approach also provides a means of detecting a partial (i.e., soft) occlusion, functioning as an accuracy control that would alert if the accuracy was deviating from the expected value.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art, including combinations of aspects or features of the embodiments and examples disclosed herein. Such other examples are intended to be within the scope of the claims if they have structural elements that do not differ from the literal language of the claims, or if they include equivalent structural elements with insubstantial differences from the literal languages of the claims. The specific embodiments described above have been shown by way of example, and it should be understood that these embodiments may be susceptible to various modifications and alternative forms, including combinations of various features and aspects of the examples or embodiments discussed herein. It should be further understood that the claims are not intended to be limited to the particular forms disclosed, but rather to cover all modifications, equivalents, and alternatives falling within the spirit and scope of this disclosure.

1. An automatic syringe pump, comprising:
a user interface comprising a syringe holder;
a mechanical power subsystem comprising a spring;
a mechanical power transfer subsystem; and
a timing subsystem comprising a ratchet and two pawl arrangement.

2. The automatic syringe pump of claim 1, wherein the user interface comprises:
a microcontroller; and
a LCD display in communication with the microcontroller.

3. The automatic syringe pump of claim 2, wherein the microcontroller is configured to store and execute one or more drug protocols.

4. The automatic syringe pump of claim 1, wherein the spring comprises a constant force spring.

5. The automatic syringe pump of claim 1, wherein the ratchet and two pawl arrangement is configured such that when one pawl is disengaged, the other pawl is engaged with the ratchet.

6. The automatic syringe pump of claim 1, wherein the ratchet and two pawl arrangement is configured such that the ratchet can only rotate half a tooth before engaging one of the two pawls.

7. The automatic syringe pump of claim 1, wherein the timing subsystem further comprises:
a microcontroller; and
a stepper motor configured to receive operational signals from the microcontroller.

8. The automatic syringe pump of claim 1, wherein the mechanical power transfer subsystem further comprises:
one or more rails;
a rack;
a pinion; and
a depressor cage.

9. The automatic syringe pump of claim 1, wherein the syringe holder is configured to receive a plurality of differently sized syringes.

10. The automatic syringe pump of claim 1, wherein the timing subsystem comprises a cam attached to a stepper motor, wherein the cam rotates to interact with flippers of a pawl shaft to periodically disengage the pawls in an alternating fashion.

11. The automatic syringe pump of claim 1, wherein the power subsystem further comprises a depression cage, a spring holder, and a spring mount which act in combination to translate force supplied by the spring into depression of a syringe.

12. The automatic syringe pump of claim 1, further comprising an occlusion detection system.

13. The automatic syringe pump of claim 12, wherein the occlusion detection system comprises a potentiometer configured to detect displacement of a depressor cage.

14. A method for depressing a syringe plunger, comprising:
applying force to a driveshaft using a spring;
rotating a pinion gear via the powered drive shaft such that the pinion gear moves along a rack; and
moving a depressor wall in response to the motion of the pinion gear along the rack, such that the depressor wall depresses a plunger of a syringe.

15. The method of claim 14, comprising regulating a rate of driveshaft rotation using an electronic timing subsystem.

16. The method of claim 14, wherein the spring is a constant force spring.

17. An automatic syringe pump, comprising:
a mechanical power supply comprising a constant force spring;
an electronic timing system comprising a microcontroller, a ratchet, and two pawls, and a stepper motor;
a mechanical power transfer system comprising rails, a rack, a pinion, and a depressor cage; and
a user interface comprising an LCD in communication with the microcontroller and a syringe tray.

18. The automatic syringe pump of claim 17, wherein the constant force spring wrapped around a main drum and a second drum, wherein the main drum is set screwed onto a drive shaft and allows the constant force spring to impart torque to the drive shaft and wherein the second drum sits off to the side of the main drum and is not set screwed onto the drive shaft but rotates around the drive shaft.

19. The automatic syringe pump of claim 18, wherein the drive shaft is allowed to rotate a fixed amount when the stepper motor causes one pawl to disengage.

20. The automatic syringe pump of claim 17, wherein both pawls are pressed against the ratchet due to torsional springs on the flippers.

21. The automatic syringe pump of claim 17, further comprising an occlusion detection system configured to stop or limit operation of the automatic syringe pump in the event that pressure increases within an IV line.

22. The automatic syringe pump of claim 21, wherein the occlusion detection system comprises a linear potentiometer configured to detect displacement of the depressor cage.
